(19) United States
(12) Patent Application Publication
(10) Pub. No.: US 2021/0187229 A1
(43) Pub. Date: Jun. 24, 2021

(54) Endotracheal Tube Mount

(71) Applicant: The 5137 Arrowhead Lakes Drive, LLC, Glendale, AZ (US)

(72) Inventor: Jugroop Brar, Glendale, AZ (US)

(73) Assignee: The 5137 Arrowhead Lakes Drive, LLC, Glendale, AZ (US)

(21) Appl. No.: 16/722,306

(22) Filed: Dec. 20, 2019

(51) Int. Cl. A61M 16/04

(52) U.S. Cl. CPC A61M 16/0493; A61M 2025/022; A61M 16/0497

(57) ABSTRACT

An endotracheal tube mount includes an armature including a central rib having opposed ends and a tube holder assembly mounted to the armature for horizontal movement along the rib between the opposed ends. The tube holder assembly includes a shuttle which rides on the central rib between the opposed ends and tube holder means which are coupled to the shuttle and are for holding an endotracheal tube in a position below the rib. In the mount, one of the armature and tube holder assembly includes vertical adjustment means for adjusting the position of the endotracheal tube vertically with respect to the rib.

### FIELD OF THE INVENTION

The present invention relates generally to medical devices, and more particularly to endotracheal tubes and accessories.

### BACKGROUND OF THE INVENTION

Oral intubation is often a necessary technique for establishing an airway in patients during surgery or other medical procedures. For prolonged intubation, an endotracheal tube is usually fastened to a patient so that it remains within the airway. The patient's mouth is typically closed, or partially closed, around the tube, such that the lips rest on the tube. This is known to cause sores or blisters on the lips. An improved device for reducing the length and location of exposure between the lips and endotracheal tube is needed.

### SUMMARY OF THE INVENTION

An endotracheal tube mount includes an armature including a central rib having opposed ends and a tube holder assembly mounted to the armature for horizontal movement along the rib between the opposed ends. The tube holder assembly includes a shuttle which rides on the central rib between the opposed ends and tube holder means which are coupled to the shuttle and are for holding an endotracheal tube in a position below the rib. In the mount, one of the armature and tube holder assembly includes vertical adjustment means for adjusting the position of the endotracheal tube vertically with respect to the rib.

The above provides the reader with a very brief summary of some embodiments described below. Simplifications and omissions are made, and the summary is not intended to limit or define in any way the scope of the invention or key aspects thereof. Rather, this brief summary merely introduces the reader to some aspects of some embodiments in preparation for the detailed description that follows.

### BRIEF DESCRIPTION OF THE DRAWINGS

Referring to the drawings:

FIGS. 1A and 1B are front perspective views of an endotracheal tube mount;

FIGS. 2 and 3 are top and bottom perspective views of the endotracheal tube mount of FIG. 1A, respectively;

FIG. 4 is a front perspective view of the endotracheal tube mount of FIG. 1A applied to a patient;

FIGS. 5A and 5B are front perspective views of an endotracheal tube mount;

FIG. 6 is a bottom perspective view of the endotracheal tube mount of FIG. 5A, respectively;

FIG. 7 is a front perspective view of the endotracheal tube mount of FIG. 5A applied to a patient;

FIGS. 8A and 8B are front perspective views of an endotracheal tube mount;

FIG. 9 is a bottom perspective view of the endotracheal tube mount of FIG. 8A, respectively;

FIG. 10 is a front perspective view of the endotracheal tube mount of FIG. 8A applied to a patient;

FIGS. 11A and 11B are front perspective views of an endotracheal tube mount;

FIG. 12 is a bottom perspective view of the endotracheal tube mount of FIG. 11A, respectively; and

FIG. 13 is a front perspective view of the endotracheal tube mount of FIG. 11A applied to a patient.

### DETAILED DESCRIPTION

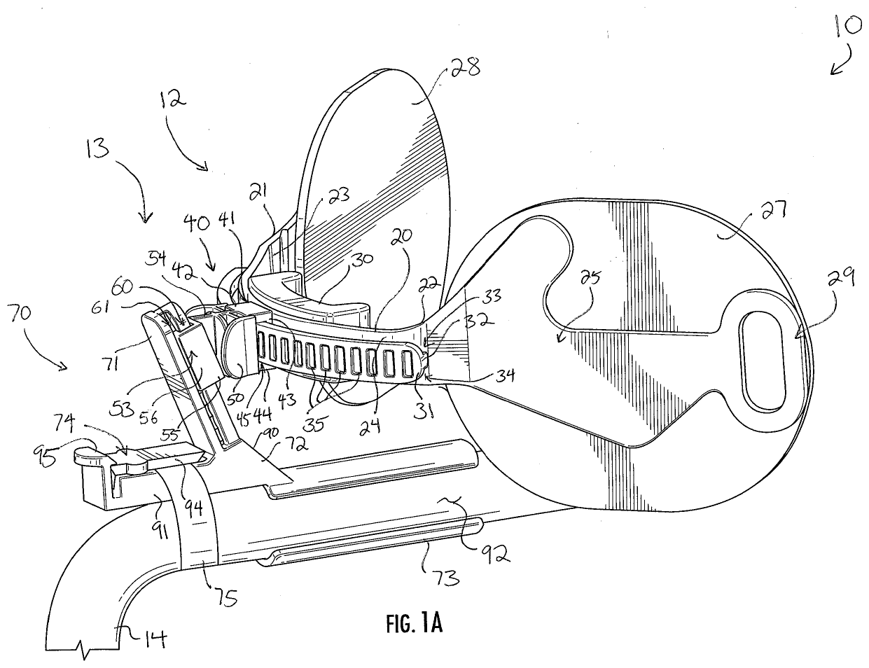

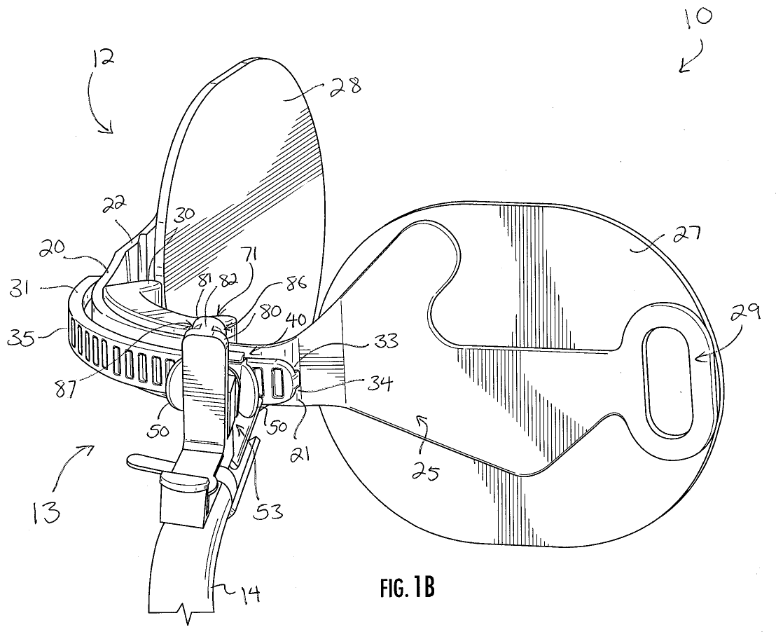

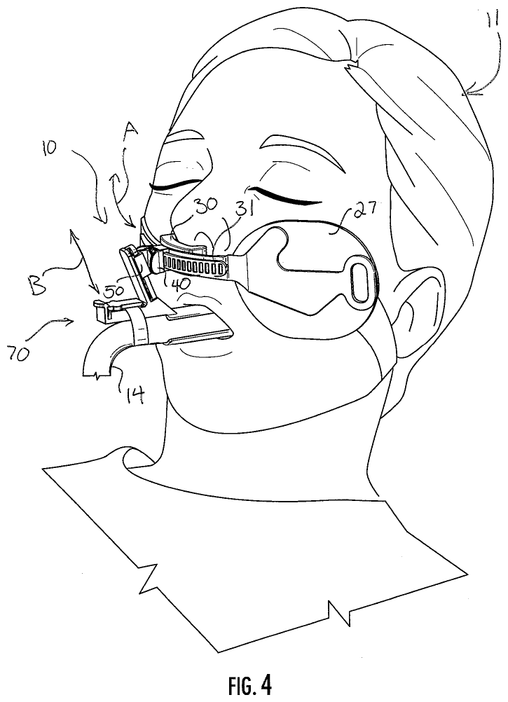

Reference now is made to the drawings, in which the same reference characters are used throughout the different figures to designate the same elements. FIGS. 1A-3 illustrate an endotracheal tube mount 10, and FIG. 4 shows that mount 10 applied to a patient 11. The mount 10 includes an armature 12 which is placed over the patient 11 and a tube holder assembly 13 which holds an endotracheal tube 14 applied to the patient 11. In this embodiment, the armature 12 has a horizontal adjustment means which moves the tube 14 horizontally across the user's mouth, and the tube holder assembly 13 has a vertical adjustment means which moves the tube 14 vertically in the user's mouth, so that the tube 14 can be moved and positioned both laterally between the user's lips and also vertically against either the upper lip or the lower lip, thereby reducing wear against the lips at any one particular location. In other embodiments, the vertical adjustment means is carried in the tube holder assembly. In this way, in all of the embodiments, at least one of the armature and tube holder assembly includes the vertical adjustment means for adjusting the position of the endotracheal tube vertically with respect to the rib.

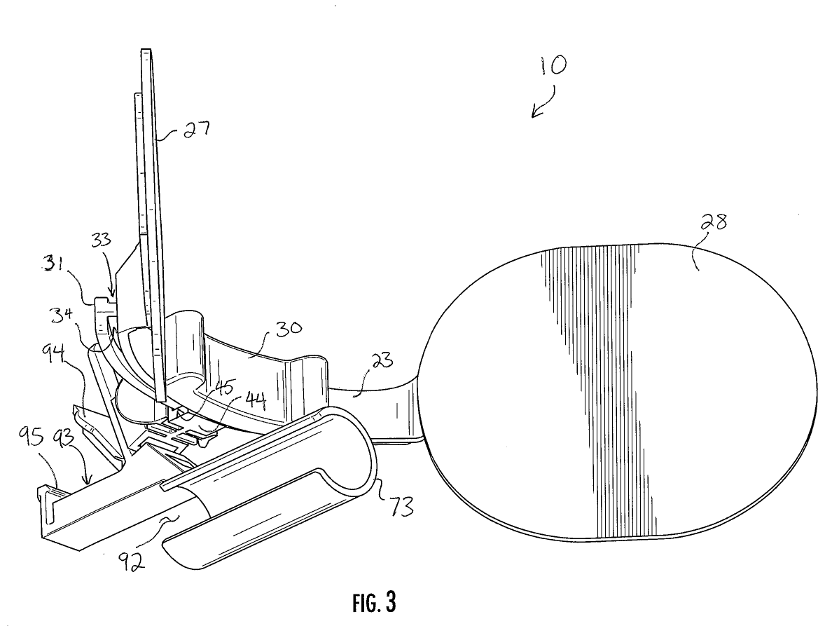

The armature 12 includes a central rib 20 having opposed ends 21 and 22 and a front 23 (shown better in FIG. 3) and an opposed back 24. The rib 20 is arcuate between the ends 21 and 22. The ends 21 and 22 are formed integrally to ears 25 and 26, to which pads 27 and 28 are affixed, respectively. The pads 27 and 28 are soft, flexible, compressive pads, and preferably have adhesive inner faces so that they can be secured on the patient 11 in operation. The ears 25 and 26 also include loops 29 to which an elastic band can be engaged, which elastic band is used to fasten the mount 10 about the head of the patient 11.

A pad 30 is carried on the front 23 of the rib 20. The pad 30 is soft and compressive; when the mount 10 is used with a patient, the pad 30 spaces the rib 20 apart from the philtrum of the patient so that the rib 20 does not rub or rest directly against the face of the patient 11. The pad 30 is preferably adhesively fixed to the front 23 of the rib 20, but may be formed integrally, sonically welded, or secured in some like fashion.

On the back 24 of the rib 20 is a track 31. The track 31 is raised off the back 24 of the rib 20 by an inner wall or web 32 extending centrally along the rib 20 along the full length of the rib 20. Because the track 31 is spaced off the back 24 of the rib 20, the inner web 32 defines opposed upper and lower channels 33 and 34 between the track 31 and the rib 20. The upper and lower channels 33 and 34 open upwardly and downwardly, respectively, and are slender channels behind the rib 20 acting as a guide, as will be explained.

A large plurality of vertical depressions 35 are formed into the track 31. These depressions 35 are discrete and spaced apart from each other, and each is generally rectangular. They extend approximately halfway into the track 30 and so are blind depressions. The upper and lower channels 33 and 34, together with the depressions 35, cooperate to engage with and allow horizontal movement of the tube holder assembly 13.

The tube holder assembly 13 holds the tube 14 in an adjustable position below the rib 20, as shown in FIG. 1A. The tube holder assembly 13 is mounted to the track 31 of the armature 12 for horizontal movement along the track 31 between the opposed ends 21 and 22 of the rib 20. The tube holder assembly 13 further includes vertical adjustment means for vertical movement of the tube 14 with respect to the rib 20. In this way, the tube holder assembly 13 positions the tube 14 bother horizontally and vertically with respect to the mouth of the patient 11.

The tube holder assembly 13 includes a shuttle 40 which rides on the rib 20 between its opposed ends 21 and 22. The shuttle 40 includes a C-shaped block having a base 42 and opposed upper and lower arms 43 and 44 which extend rectilinearly forward and then toward each other, so as to define an open slot 45. Opposite the base 42, the upper and lower arms 43 and 44 do not contact each other; rather, their ends are slightly spaced apart from each other to form a gap (not shown) in communication with the slot 45. When the shuttle 40 is fit to the track 31, the base 42 rides just above the depressions 35 in the track 31, the upper and lower arms 43 and 44 each extend around the track 31, and the gap between the arms 43 and 44 accommodates the web 32 connecting the track 31 to the rib 20. When the shuttle 40 moves horizontally along the track 31, the upper arm 43 and lower arm 44 slide within the upper channel 33 and lower channel 34, respectively, and the gap between the upper and lower arms 43 and 44 slides over the web 32. This interaction guides the movement of the shuttle 40 and prevents the shuttle 40 from inadvertently coming free of the rib 20.

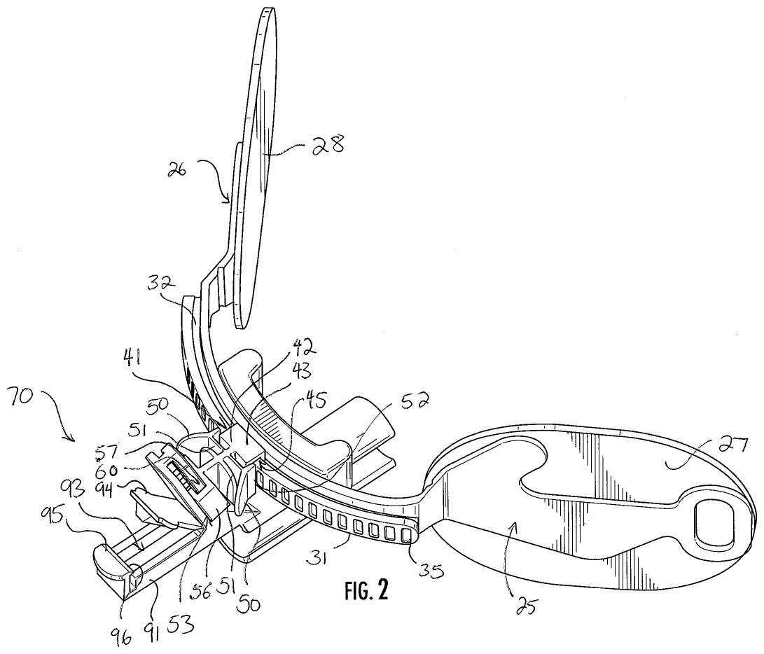

The shuttle 40 includes tabs 50 for locking the position of the shuttle 40 along the track 31. The tabs 50, best shown in FIG. 2, are on opposed sides of the shuttle 40. They extend outward from the shuttle 40, diagonally away from the track 31, and are biased forwardly toward the track 31 on sprung, flexible arms 51. The tabs 50 have forwardly-directed teeth 52 which move into and out of the depressions 35 in the track to engage with them. The tabs 50 are biased forwardly, and when the tabs 50 are forward, the teeth 52 engage with the depressions 35 and prevent lateral movement of the shuttle 40 along the track 31. Because the depressions 35 are discrete and spaced-apart, engagement of the teeth 52 in the depressions 35 indexes the shuttle 40 along the track 31. Squeezing the tabs 50 backward and toward each other causes the tabs 50 to pivot away from the track 31 and the teeth 52 to move out of the depressions 35, thereby breaking the engagement of the tabs 50 and depressions 35, so that the shuttle 40 may be freely moved along the track 31.

Referring now to FIGS. 1A and 2, a slide 53 is coupled to the shuttle 40 and constitutes part of the tube holder assembly 13. A short connecting web 54 extends from the base 42 of the shuttle 40 to the slide 53. The slide 53 is similar in construction to the block 41 of the shuttle 40: it includes a base 55 and two arms 56 and 57 which extend rectilinearly rearward and then toward each other, so as to define an open slot 60. Opposite the base 55, the arms 56 and 57 do not contact each other; rather, their free ends are slightly spaced apart from each other to form a gap 61 in communication with the slot 60. The slot 60 and gap 61 receive a track of a tube holder means 70 which holds the tube 14 in position below the rib 20; the slide 53 couples the tube holder means 70 to the shuttle 40 of the armature 12.

The tube holder means 70 includes a vertical guide 71 extending upward from a base 72, a clamp 73 projecting forward from the base 72 to hold the tube 14, and a crimp 74 for securing a strap 75 around the tube 14. Turning now primarily to FIG. 1B, the vertical guide 71 includes a base 80, a track 81, and a web 82 extending transversely to the base 80 and track 81, connecting the base 80 to the track 81 and spacing the two slightly apart from each other. Because the track 81 is spaced apart from the base 80 by the web 82, the web 82 defines opposed, separate, lateral channels 86 and 87 between the track 81 and the base 80. The channels 86 and 87 open laterally, and are slender channels acting as guides. When the slide 53 is fit to the vertical guide 71, the base 55 of the slide 53 rides just off the track 81, and the arms 56 and 57 each extend around the track 81 into the channels 86 and 87, respectively. The gap 61 between the arms 56 and 57 accommodates the web 82 in the guide 71. When the slide 53 moves vertically along the vertical guide 71, the arms 56 and 57 slide within the channels 86 and 87, respectively, and the gap 61 slides over the web 82. This interaction guides the vertical movement of the slide 53 and prevents the slide 53 from inadvertently coming free of the vertical guide 71. As such, the slide 53 and the vertical guide 71 cooperate to define the vertical adjustment means of the tube holder assembly 13.

In a preferred embodiment, as can be seen in FIGS. 1A and 2, the web 82 has ribbed lateral surfaces and the terminal edges of the arms 56 and 57 are complementally ribbed, such that the complemental ribs engage with each other so as to lock the slide 53 in indexed vertical positions along the vertical guide 71.

The vertical guide 71 extends upward from the base 72, to which it is integrally and monolithically formed. The base is a short, solid body having a roughly rectangular cross-section. It has a front 90 and a rear 91. The clamp 73 is formed integrally and monolithically to the front 90 as an extension thereof. The clamp 73 is a cylindrical clamp, being a severed hollow tube with an elongate opening 92 extending parallel to its axis. The opening 92 is approximately the same dimension as the radius of the cylindrical clamp 73, and the clamp 73 preferably has an inner diameter which is just smaller than the outer diameter of the tube 14. The clamp 73 is constructed from a pliant, flexible, and resilient material or combination of materials, such as plastic, so that the opening 92 can be enlarged to pass the tube 14 through the opening 92. The opening 92 of the clamp 73 then returns to its previous dimension, smaller than the diameter of the tube 14, and the tube 14 is snugly retained in the clamp 73. This snug retention holds the tube 14 and prevents relevant axial and rotational movement of the tube with respect to the clamp 73.

The tube 14 is also secured by the strap 75 in the crimp 74. The strap 75 is a thin, flexible ribbon or length of plastic. The rear 91 of the tube holder means 70 includes a solid rectangular prismatic body with an elongate socket 93 (best seen in FIG. 2) oriented with an upward opening behind the vertical guide 71. The socket 93 is open, but an arm 94 above the socket 93 is hinged to pivot between an unlocked position (as shown in FIGS. 2 and 3) and a locked position (as shown in FIGS. 1A and 1B). The socket 93 and arm 94 cooperate to define the crimp 74. The arm 94 is hinged to the body proximate the bottom of the vertical guide 71, and so its free end projects rearwardly away from the tube holder means 70. In the unlocked position, the arm 94 is up, out of the socket 93, and the strap 75 may be placed into the socket 93 or removed therefrom. In the locked position, the arm 94 is depressed into the socket 93, where it may capture the strap 75. The socket 93 has a triangular-shaped cross-section, and the underside of the arm 94 has a complemental, triangular, downward projection which snaps into the socket 93. When the strap 75 is placed into the socket 93 and the arm 94 is closed in the socket 93 in the locked position, the strap 75 is crimped in the socket 93 and thus retained therein. Further reinforcing this retention is a catch 95 formed at the end of the tube holder means 70. The catch 95 is a lip that hangs over the socket 93. The catch 95 has a sloped upper face 96; when the arm 94 is moved downward toward the locked position, the arm 94 encounters the sloped upper face 96, pushes the catch 95 out of the way, and the arm 94 snaps under the catch 95. The catch 95 returns to its previous position and prevents the arm 94 from inadvertently rising upward out of the locked position. The arm 94 is snugly retained between the top of the socket 93 and the underside of the catch 95. When the arm 94 is to be moved out of the locked position, the back of the catch 95 is depressed, which pivots the catch 95 backward and allows the arm 94 to clear the catch 95.

Referring to FIG. 4, in operation, the mount 10 is useful for holding the tube 14 in the mouth of a patient 11 and allowing the tube 14 to be moved into a variety of locations. The pads 27 and 28 are adhesively fixed to the cheeks of the patient 11 so as to position the pad 30 roughly at the philtrum. The tube 14 is inserted into the mouth and throat of the patient 11, but can be moved. By depressing the tabs 50, the shuttle 40 is disengaged with the depressions 35 in the track 31 and can be slid laterally across the track 31, along the double-arrowed, arcuate line A. This allows a health worker to locate the tube 14 at either of the corners of the mouth, centrally in the mouth, or in some location therebetween. Moreover, the tube holder means 70 can be moved up and down on the slide 53 along the double-arrowed, arcuate line B, so that a health worker can place the tube 14 more against the upper lip or more against the lower lip. For example, if a health worker notices that the right side of the mouth of the patient 11 is becoming irritated such that a sore may develop, the health worker can slide the shuttle 40 to the left so as to position the tube 14 at the left side of the mouth (as in FIG. 1B). If the health worker then notices that the upper left side of the mouth is developing a sore, then the worker can move the slide 53 up on the vertical guide 71, such that the tube 14 moves down against the lower lip (as in FIG. 4).

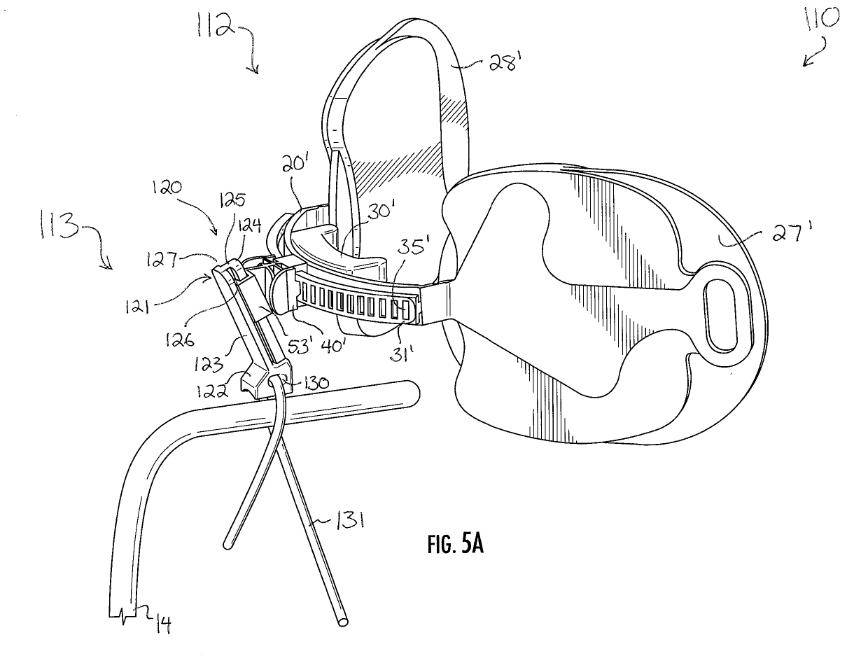

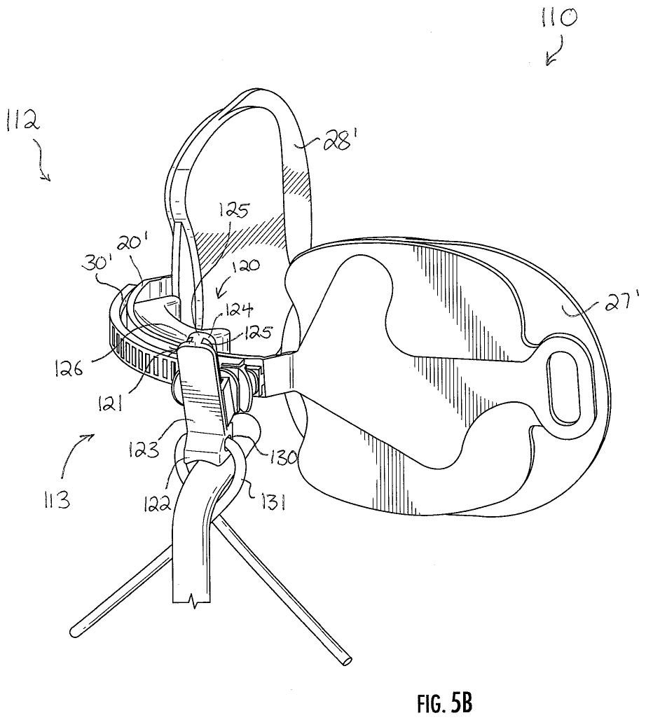

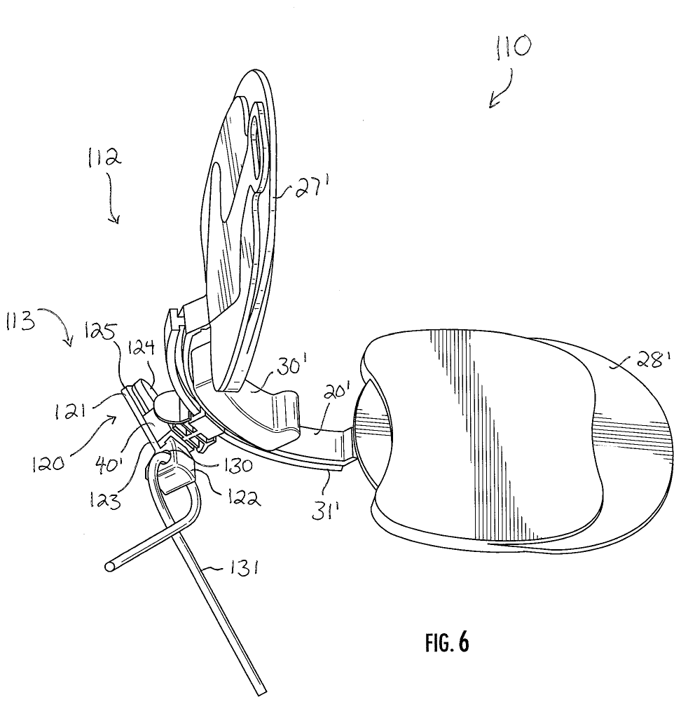

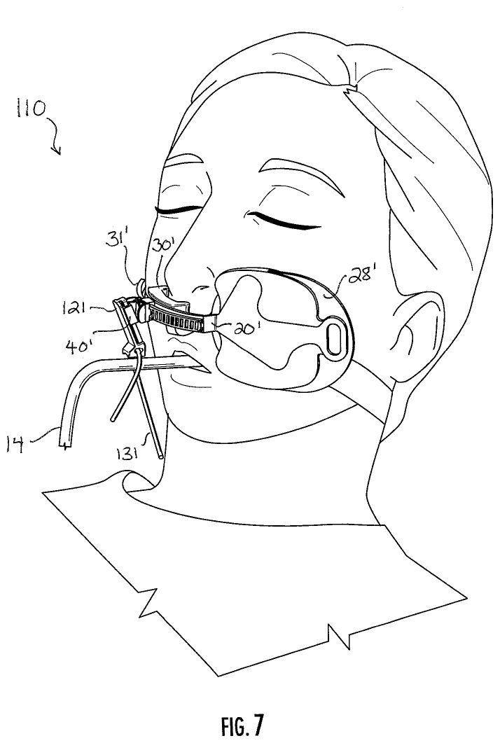

FIGS. 5A-6 illustrate an endotracheal tube mount 110, and FIG. 7 shows that mount 110 applied to the patient 11. The mount 110 includes an armature 112 which is placed over the patient 11 and a tube holder assembly 113 which holds the endotracheal tube 14 applied to the patient 11. In this embodiment, the armature 112 has a horizontal adjustment means which moves the tube 14 horizontally across the user's mouth, and the tube holder assembly 113 has a vertical adjustment means which moves the tube 14 vertically in the user's mouth, so that the tube 14 can be moved and positioned both laterally between the user's lips and also vertically against either the upper lip or the lower lip, thereby reducing wear against the lips at any one particular location.

Many of the structural elements and features of the mount 110 are identical to those of the mount 10. As such, the same names and references characters are used to identify such common structural elements and features, with those of the mount 110 being marked with a prime (“′”) symbol to distinguish them from those of the mount 10. Moreover, common structural elements and features of the mount 110 may not be described in detail below for simplicity and may not be illustrated in the drawings. The reader will understand the structure, arrangement, and function of the common structural elements and features from the description of them with respect to the mount 10. For example, the armature 112 includes a rib 20′, ends 21′ and 22′, front 23′, back 24′, ears 25′ and 26′, pads 27′ and 28′, pad 30′, track 31′, web 32′, upper and lower channels 33′ and 34′, and depressions 35′.

The tube holder assembly 113 is different from the tube holder assembly 13 of the mount 10. It includes the shuttle 40′, block 41′, base 42′, upper and lower arms 43′ and 44′, slot 45′, tabs 50′, arms 51′, teeth 52′, slide 53′, base 55′, arms 56′ and 57′, slot 60′, and gap 61′, but it has a different tube holder means 120. The slot 60′ and gap 61′ receive a track of a tube holder means 120 which holds the tube 14 in position below the rib 20′; the slide 53′ couples the tube holder means 120 to the shuttle 40′ of the armature 112.

The tube holder means 120 includes a vertical guide 121 extending upward from a base 122 to which a tie 131 is fastened for securing the tube 14. The vertical guide 121 includes a base 123, a track 124, and a web 125 extending transversely to the base 123 and track 124, connecting the base 123 to the track 124 and spacing the two slightly apart from each other. Because the track 124 is spaced apart from the base 123 by the web 125, the web 125 defines opposed, separate, lateral channels 126 and 127 between the track 124 and the base 123. The channels 126 and 127 open laterally, and are slender channels acting as guides. When the slide 53′ is fit to the vertical guide 121, the base 55′ of the slide 53s rides just off the track 124, and the arms 56′ and 57′ each extend around the track 124 into the channels 126 and 127, respectively. The gap 61′ between the arms 56′ and 57′ accommodates the web 125 in the guide 121. When the slide 53′ moves vertically along the vertical guide 121, the arms 56′ and 57′ slide within the channels 126 and 127, respectively, and the gap 61′ slides over the web 125. This interaction guides the vertical movement of the slide 53′ and prevents the slide 53′ from inadvertently coming free of the vertical guide 121. As such, the slide 53′ and the vertical guide 121 cooperate to define the vertical adjustment means of the tube holder assembly 113.

The vertical guide 121 extends upward from the base 122, to which it is integrally and monolithically formed. The base 122 is a short, solid body having a roughly rectangular cross-section. It is formed with a hole 130 extending laterally through the body. A tie 131 is passed through the hole 130 for wrapping around the tube 14. The tie 131 is flexible, malleable, and durable, and holds the shape in which it is arranged. As such, the tie 131 is wrapped around the tube 14 to hold the tube 14 closely to the base 122. The base 122 has a concave underside to accommodate the curvature of the tube 14.

Referring to FIG. 7, in operation, the mount 110 is useful for holding the tube 14 in the mouth of a patient 11 and allowing the tube 14 to be moved into a variety of locations. The pads 27′ and 28′ are adhesively fixed to the cheeks of the patient 11 so as to position the pad 30′ roughly at the philtrum. The tube 14 is inserted into the mouth and throat of the patient 11, but can be moved. By depressing the tabs 50′, the shuttle 40′ is disengaged with the depressions 35′ in the track 31′ and can be slid laterally across the track 31′. This allows a health worker to locate the tube 14 at either of the corners of the mouth, centrally in the mouth, or in some location therebetween. Moreover, the tube holder means 120 can be moved up and down on the slide 53′, so that a health worker can place the tube 14 more against the upper lip or more against the lower lip. For example, if a health worker notices that the right side of the mouth of the patient 11 is becoming irritated such that a sore may develop, the health worker can slide the shuttle 40′ to the left so as to position the tube 14′ at the left side of the mouth (as in FIG. 5B). If the health worker then notices that the upper left side of the mouth is developing a sore, then the worker can move the slide 53′ up on the vertical guide 121, such that the tube 14 moves down against the lower lip (as in FIG. 7).

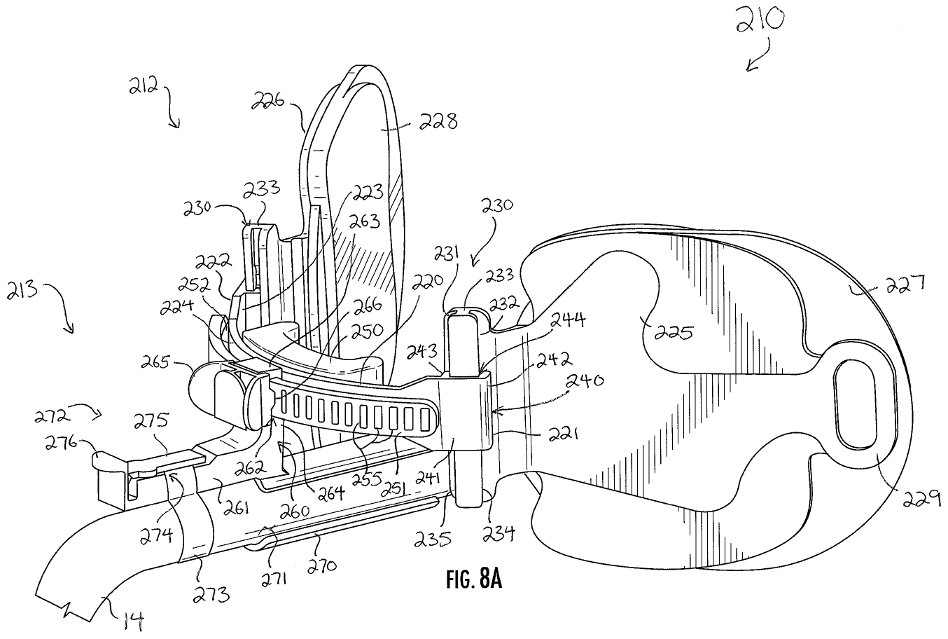

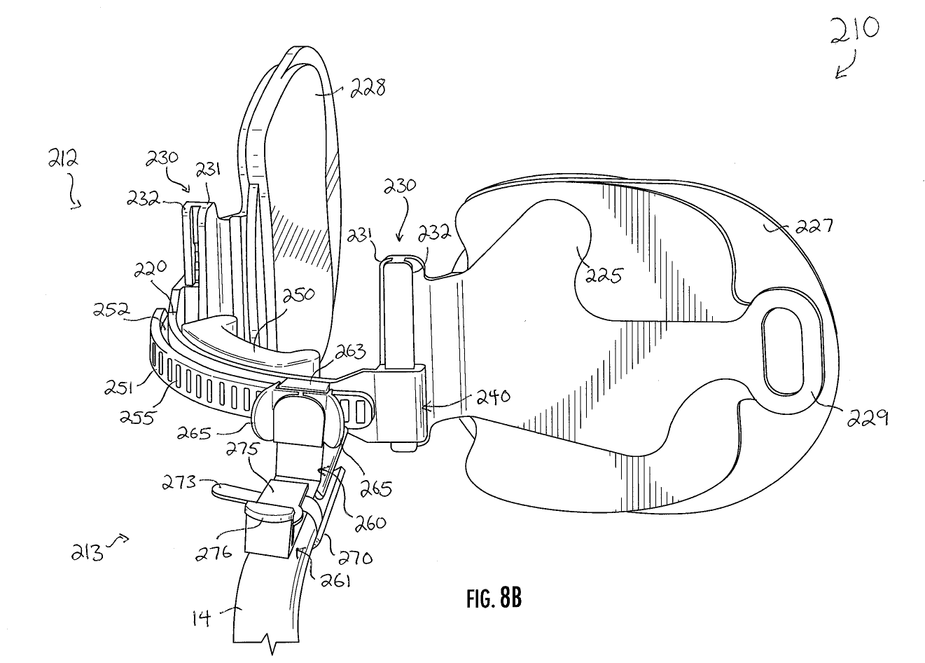

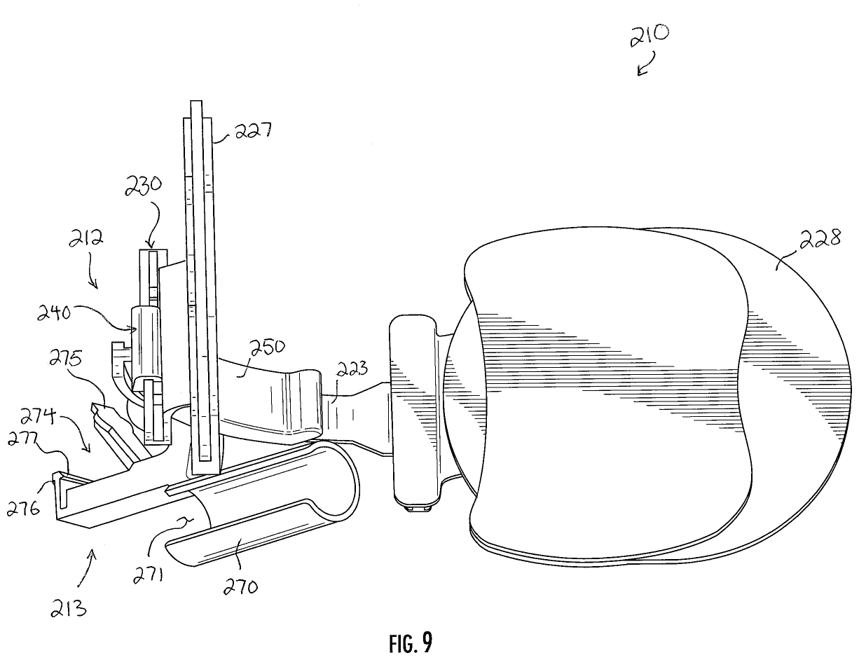

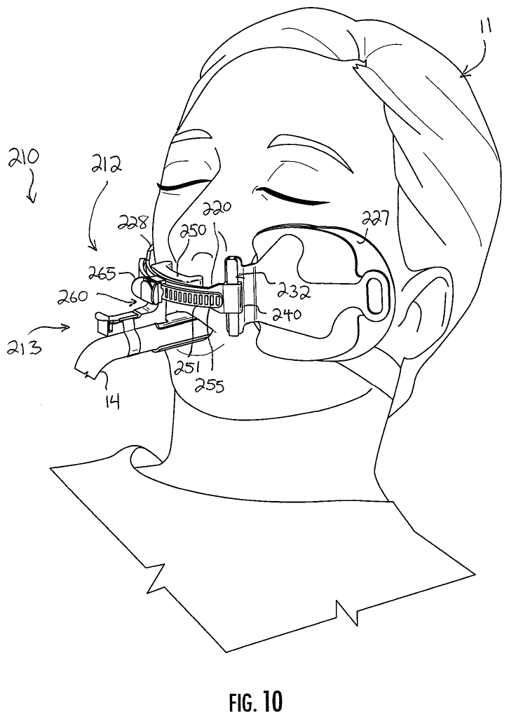

FIGS. 8A-9 illustrate an endotracheal tube mount 210, and FIG. 10 shows that mount 210 applied to a patient 11. The mount 210 includes an armature 212 which is placed over the patient 11 and a tube holder assembly 213 which holds an endotracheal tube 14 applied to the patient 11. In this embodiment, the armature 212 has a horizontal adjustment means which moves the tube 14 horizontally across the user's mouth, and it also has a vertical adjustment means which moves the tube 14 vertically in the user's mouth. In this way, the armature 212 allows the tube 14 to be moved and positioned both laterally between the user's lips and also vertically against either the upper lip or the lower lip, thereby reducing wear against the lips at any one particular location.

The armature 212 includes a central rib 220 having opposed ends 221 and 222 and a front 223 (shown better in FIG. 9) and an opposed back 224. The rib 220 is arcuate between the ends 221 and 222. The ends 221 and 222 terminate in tracks, which are coupled to ears 225 and 226, to which pads 227 and 228 are affixed, respectively. The pads 227 and 228 are soft, flexible, compressive pads, and preferably have adhesive inner faces so that they can be secured on the patient 11 in operation. The ears 225 and 226 also include loops 229 to which an elastic band can be engaged, which elastic band is used to fasten the mount 210 about the head of the patient 11.

The ends 221 and 222 of the and the ears 225 and 226 are coupled to each other to form the vertical adjustment means of the armature 212. There are two identical vertical adjustment means, one located at each end 221 and 222, and so only one will be discussed with the understanding that the description applies equally to both. The ear 225 terminates at its inner end with a vertical guide 230 including an elongate base 231, a coextensive track 232, and a web 233 extending transversely between the base 231 and track 232, connecting the base 231 and track 232 and spacing them slightly apart from each other. Because the track 232 is spaced apart from the base 231 by the web 233, the web 233 defines opposed, separate, lateral channels 234 and 235 between the track 232 and the base 231. The channels 234 and 235 open vertically to the sides, and are slender channels acting as guides for a slide 240.

The slide 240 includes a short base 241 and two arms 242 and 243 which extend rectilinearly rearward and then toward each other, so as to define an open slot 244 between the arms 242 and 243 and the base 241. Opposite the base 241, the arms 242 and 243 do not contact each other; rather, their free ends are slightly spaced apart from each other to form a gap (not shown) in communication with the slot 244. The slot 244 and gap receive the track 232 of the vertical guide 230 when the slide 240 is fit onto the vertical guide 230. When the slide 240 is applied to the vertical guide 230, the base 231 of the slide 240 rides just off the track 232, and the arms 242 and 243 each extend around the track 232 into the channels 234 and 235, respectively. The gap between the arms 242 and 243 accommodates the web 233 in the guide 230. When the slide 240 moves vertically along the vertical guide 230, the arms 242 and 243 slide within the channels 234 and 235, respectively, and the gap slides over the web 233. This interaction guides the vertical movement of the slide 240 and prevents the slide 240 from inadvertently coming free of the vertical guide 230. As such, the vertical guide 230 and slide 240 mounted thereto cooperate to define the vertical adjustment means of the armature 212. And, as noted above, both ends 221 and 222 of the rib 220 include identical structure.

In a preferred embodiment, as can be seen in the vertical guide 230 proximate the end 222 in FIG. 8A, the web 233 has ribbed lateral surfaces. Preferably, the terminal edges of the arms 242 and 243 are complementally ribbed, such that the complemental ribs engage with each other so as to lock the slide 240 in indexed vertical positions along the vertical guide 230.

A pad 250 is carried on the front 223 of the rib 220. The pad 250 is soft and compressive; when the mount 210 is used with a patient, the pad 250 spaces the rib 220 apart from the philtrum of the patient so that the rib 220 does not rub or rest directly against the face of the patient 11. The pad 250 is preferably adhesively fixed to the front 223 of the rib 220, but may be formed integrally, sonically welded, or secured in some like fashion to the rib 220.

On the back 224 of the rib 220 is a track 251. The track 251 is raised off the back 224 of the rib 220 by an inner wall or web 252 extending centrally along the rib 220 along the full length of the rib 220. Because the track 251 is spaced off the back 224 of the rib 220, the inner web 252 defines opposed upper and lower channels 253 and 254 between the track 251 and the rib 220. The upper and lower channels 253 and 254 open upwardly and downwardly, respectively, and are slender channels behind the rib 220 acting as a guide, as will be explained.

A large plurality of vertical depressions 255 are formed into the track 251. These depressions 255 are discrete and spaced apart from each other, and each is generally rectangular. They extend approximately halfway into the track 250 and so are blind depressions. The upper and lower channels 253 and 254, together with the depressions 255, cooperate to engage with and allow horizontal movement of the tube holder assembly 213.

The tube holder assembly 213 is a tube holder means, structured and arranged to hold the tube 14 in an adjustable position below the rib 220, as shown in FIG. 8A. The tube holder assembly 213 is mounted to the track 251 of the armature 212 for horizontal movement along the track 251 between the opposed ends 221 and 222 of the rib 220. The tube holder assembly 213 includes a body with an upper portion 260 and a lower portion 261. The upper and lower portions 260 and 261 are integrally and monolithically formed. The upper portion 260 extends generally downward away from the rib 251, and the lower portion 261 extends rearwardly away from the bottom of the upper portion 260. The upper portion 260 rides on the rib 220 between its opposed ends 221 and 222.

The upper portion 260 includes an open slot 262 that receives the track 251. The upper portion 260 includes a gap (not shown) which is a thin break or sever in the upper portion 260 in communication with the slot 262; the gap accommodates and rides over the web 252 connecting the track 251 to the rib 220. In this way, the upper portion 260 defines a shuttle, somewhat similar to the shuttle 40 in the mount 10. The upper portion 260 has an upper arm 263 and a lower arm 264 extending around and defining the slot 262. The free ends of the upper and lower arms are spaced apart by the gap.

When the upper portion 260 is fit to the track 251, it rides just above the depressions 255 in the track 251, and when the upper portion 260 moves horizontally along the track 251, the upper arm 263 and lower arm 264 slide within the upper channel 253 and lower channel 354, respectively, and the gap between the upper and lower arms 263 and 264 slides over the web 252. This interaction guides the movement of the upper portion 260 and prevents the upper portion 260 from inadvertently coming free of the rib 220.

The upper portion 260 includes tabs 265 for locking the position of the upper portion 260 along the track 251. The tabs 265, best shown in FIG. 8B, are on opposed sides of the upper portion 260. They extend outward from the upper portion 260, diagonally away from the track 251, and are biased forwardly toward the track 251 on sprung, flexible arms. The tabs 265 have forwardly-directed teeth 266 which move into and out of the depressions 255 in the track to engage with them. The tabs 265 are biased forwardly, and when the tabs 265 are forward, the teeth 266 engage with the depressions 255 and prevent lateral movement of the upper portion 260 along the track 251. Because the depressions 255 are discrete and spaced-apart, engagement of the teeth 266 in the depressions 255 indexes the upper portion 260 along the track 251. Squeezing the tabs 265 backward and toward each other causes the tabs 265 to pivot away from the track 251 and the teeth 266 to move out of the depressions 255, thereby breaking the engagement of the tabs 265 and depressions 255, so that the upper portion 260 may be freely moved along the track 232.

The lower portion 261 of the tube holder assembly 213 is formed to the upper portion 260 and extends below and rearward from the base of the upper portion 260. The rear portion 261 includes a clamp 270 projecting forwardly to hold the tube 14, and a crimp 272 for securing a strap 273 around the tube 14. The clamp 270 projects forwardly from the base of the lower portion 261 of the tube holder assembly 231. The clamp 270 is formed integrally and monolithically to the lower portion 261 as an extension thereof. The clamp 270 is a cylindrical clamp, being a severed hollow tube with an elongate opening 271 extending parallel to its axis. The opening 271 is approximately the same dimension as the radius of the cylindrical clamp 270, and the clamp 270 preferably has an inner diameter which is just smaller than the outer diameter of the tube 14. The clamp 270 is constructed from a pliant, flexible, and resilient material or combination of materials, such as plastic, so that the opening 271 can be enlarged to pass the tube 14 through the opening 271. The opening 271 of the clamp 270 then returns to its previous dimension, smaller than the diameter of the tube 14, and the tube 14 is snugly retained in the clamp 270. This snug retention holds the tube 14 and prevents relevant axial and rotational movement of the tube with respect to the clamp 270.

The tube 14 is also secured by the strap 273 in the crimp 272. The strap 273 is a thin, flexible ribbon or length of plastic. The lower portion 261 includes a solid rectangular prismatic body with an elongate socket 274 (similar to the socket 93 of the mount 10) oriented with an upward opening behind the upper portion 260. The socket 274 is open, but an arm 275 above the socket 274 is hinged to pivot between an unlocked position (as shown in FIG. 9) and a locked position (as shown in FIGS. 8A, 8B, and 10). The socket 274 and arm 275 cooperate to define the crimp 272. The arm 275 is hinged to the body of the tube holder assembly 213 proximate the base of the upper portion 260, and so its free end projects rearwardly away from the upper portion 260. In the unlocked position, the arm 275 is up, out of the socket 274, and the strap 273 may be placed into the socket 274 or removed therefrom. In the locked position, the arm 275 is depressed into the socket 274, where it may capture the strap 273. The socket 274 has a triangular-shaped cross-section (identical to that of the socket 93), and the underside of the arm 275 has a complemental, triangular, downward projection which snaps into the socket 274. When the strap 273 is placed into the socket 274 and the arm 275 is closed in the socket 274 in the locked position, the strap 273 is crimped in the socket 274 and thus retained therein. Further reinforcing this retention is a catch 276 formed at the end of the tube holder means 70. The catch 276 is a lip that hangs over the socket 274. The catch 276 has a sloped upper face 277; when the arm 275 is moved downward toward the locked position, the arm 275 encounters the sloped upper face 277, pushes the catch 276 out of the way, and the arm 275 snaps under the catch 276. The catch 276 returns to its previous position and prevents the arm 275 from inadvertently rising upward out of the locked position. The arm 275 is snugly retained between the top of the socket 274 and the underside of the catch 276. When the arm 275 is to be moved out of the locked position, the back of the catch 276 is depressed, which pivots the catch 276 backward and allows the arm 275 to clear the catch 276.

Referring to FIG. 10, in operation, the mount 210 is useful for holding the tube 14 in the mouth of a patient 11 and allowing the tube 14 to be moved into a variety of locations. The pads 227 and 228 are adhesively fixed to the cheeks of the patient 11 so as to position the pad 250 roughly at the philtrum. The tube 14 is inserted into the mouth and throat of the patient 11, but can be moved. By depressing the tabs 265, the upper portion 260 is disengaged with the depressions 255 in the track 251 and can be slid laterally across the track 251, thereby moving the tube holder assembly 213 horizontally. This allows a health worker to locate the tube 14 at either of the corners of the mouth, centrally in the mouth, or in some location therebetween.

Moreover, the rib 220 can be moved up and down on its two slides 240 fit in the tracks 232, so that a health worker can place the tube 14 more against the upper lip or more against the lower lip. For example, if a health worker notices that the right side of the mouth of the patient 11 is becoming irritated such that a sore may develop, the health worker can slide the upper portion 260 to the left so as to position the tube 14 at the left side of the mouth (as in FIG. 8B). If the health worker then notices that the lower left side of the mouth is developing a sore, then the worker can move the slides 240 up on the vertical guides 230, such that the tube 14 moves up against the upper lip.

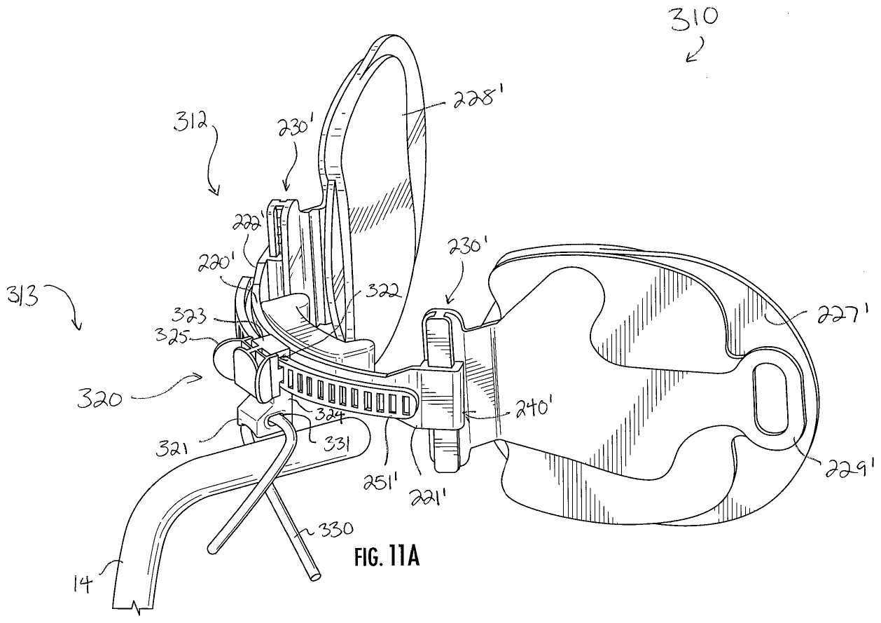

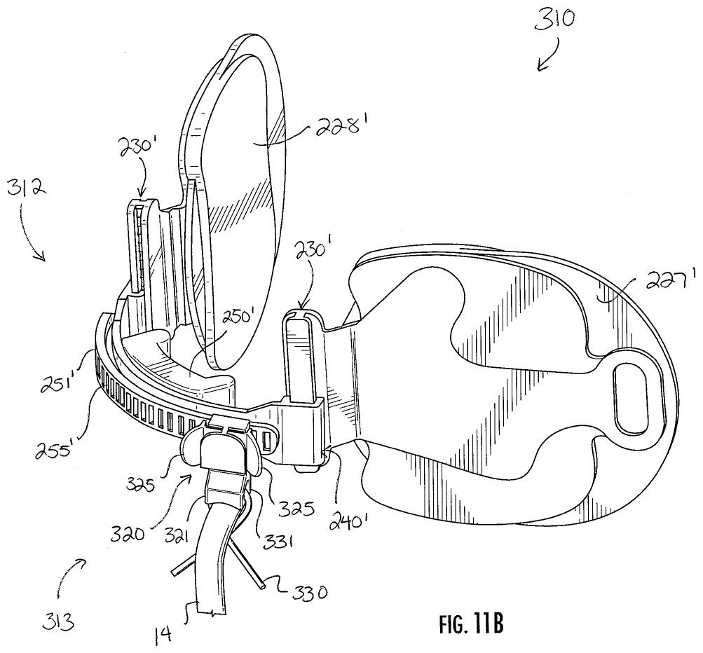

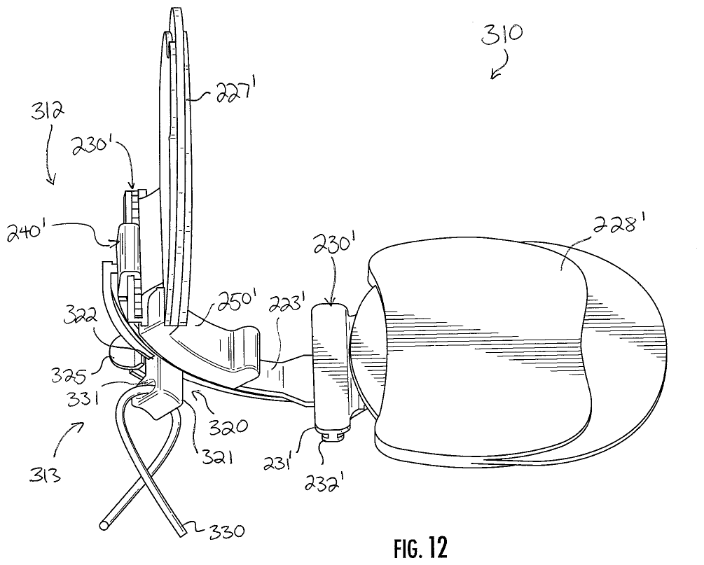

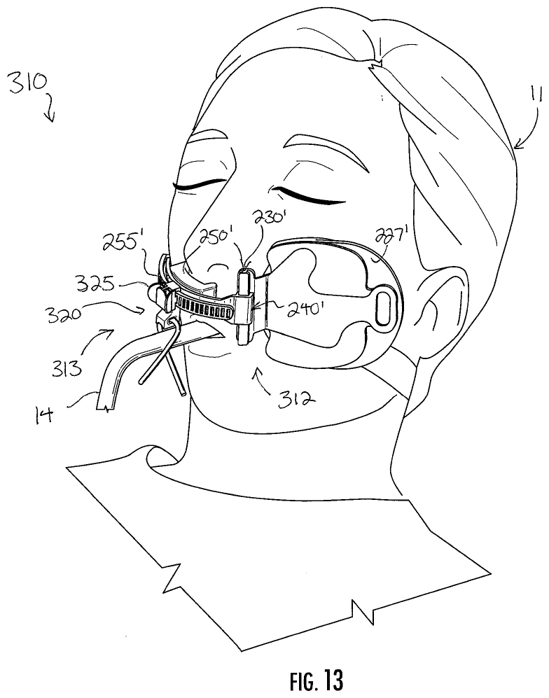

FIGS. 11A-12 illustrate an endotracheal tube mount 310, and FIG. 13 shows that mount 310 applied to a patient 11. The mount 310 includes an armature 312 which is placed over the patient 11 and a tube holder assembly 313 which holds an endotracheal tube 14 applied to the patient 11. In this embodiment, the armature 312 has a horizontal adjustment means which moves the tube 14 horizontally across the user's mouth, and it also has a vertical adjustment means which moves the tube 14 vertically in the user's mouth. In this way, the armature 312 allows the tube 14 to be moved and positioned both laterally between the user's lips and also vertically against either the upper lip or the lower lip, thereby reducing wear against the lips at any one particular location.

Many of the structural elements and features of the mount 310 are identical to those of the mount 110. As such, the same names and references characters are used to identify such common structural elements and features, with those of the mount 310 being marked with a prime (“′”) symbol to distinguish them from those of the mount 110. Moreover, common structural elements and features of the mount 310 may not be described in detail below for simplicity and may not be illustrated in the drawings. The reader will understand the structure, arrangement, and function of the common structural elements and features from the description of them with respect to the mount 10. For example, the armature 312 includes a rib 220′, ends 221′ and 222′, front 223′, back 224′, ears 225′ and 226′, pads 227′ and 228′, loops 229′, vertical guides 230′, base 231′, track 232′, web 233′, channels 234′ and 235′, slide 240′, base 241′, arms 242′ and 243′, slot 244′, pad 250′, track 251′, web 252′, upper and lower channels 253′ and 254′, and depressions 255.

The tube holder assembly 313 is different from the tube holder assembly 213 of the mount 210. The tube holder assembly 313 is a tube holder means, structured and arranged to hold the tube 14 in an adjustable position below the rib 220′, as shown in FIG. 11A. The tube holder assembly 313 is mounted to the track 251′ of the armature 312 for horizontal movement along the track 251′ between the opposed ends 221′ and 222′ of the rib 220′. The tube holder assembly 313 includes a body with an upper portion 320. The upper portion 320 extends generally downward away from the rib 251, terminating in a base 321. The upper portion 320 rides on the rib 220′ between its opposed ends 221′ and 222′.

The upper portion 320 includes an open slot 322 that receives the track 261′. The upper portion 320 includes a gap (not shown) which is a thin break or sever in the upper portion 320 in communication with the slot 322; the gap accommodates and rides over the web 252′ connecting the track 251′ to the rib 220′. In this way, the upper portion 320 defines a shuttle, somewhat similar to the shuttle 40 in the mount 10. The upper portion 320 has an upper arm 323 and a lower arm 324 extending around and defining the slot 322. The free ends of the upper and lower arms are spaced apart by the gap.

When the upper portion 320 is fit to the track 251, it rides just above the depressions 255 in the track 251, and when the upper portion 320 moves horizontally along the track 251, the upper arm 323 and lower arm 324 slide within the upper channel 253′ and lower channel 254′, respectively, and the gap between the upper and lower arms 323 and 324 slides over the web 252s. This interaction guides the movement of the upper portion 320 and prevents the upper portion 320 from inadvertently coming free of the rib 220′.

The upper portion 320 includes tabs 325 for locking the position of the upper portion 320 along the track 251′. The tabs 325, best shown in FIG. 11B, are on opposed sides of the upper portion 320. They extend outward from the upper portion 320, diagonally away from the track 251′, and are biased forwardly toward the track 251′ on sprung, flexible arms. The tabs 325 have forwardly-directed teeth 326 which move into and out of the depressions 255′ in the track to engage with them. The tabs 325 are biased forwardly, and when the tabs 325 are forward, the teeth 326 engage with the depressions 255′ and prevent lateral movement of the upper portion 320 along the track 251′. Because the depressions 255′ are discrete and spaced-apart, engagement of the teeth 326 in the depressions 255′ indexes the upper portion 320 along the track 251′. Squeezing the tabs 325 backward and toward each other causes the tabs 325 to pivot away from the track 251′ and the teeth 326 to move out of the depressions 255′, thereby breaking the engagement of the tabs 325 and depressions 255′, so that the upper portion 320 may be freely moved along the track 232′.

The base 321 of the tube holder assembly 313 is formed to the upper portion 320 and extends below and just slightly rearward from the upper portion 320. A tie 330 is fastened to the base 321 for securing the tube 14. A hole 331 is formed entirely through the base 321 laterally. The tie 330 is constructed from a flexible, malleable, and durable material, and it holds the shape into which it is arranged. The tie 330 is passed through the hole 331 and then wrapped around the tube 14 under the base 321 of the tube holder assembly 313 to hold the tube 14 closely to the base 321. The base 321 has a concave underside to accommodate the curvature of the tube 14.

Referring to FIG. 13, in operation, the mount 310 is useful for holding the tube 14 in the mouth of a patient 11 and allowing the tube 14 to be moved into a variety of locations. The pads 227′ and 228′ are adhesively fixed to the cheeks of the patient 11 so as to position the pad 250′ roughly at the philtrum. The tube 14 is inserted into the mouth and throat of the patient 11, but can be moved. By depressing the tabs 325, the upper portion 320 is disengaged with the depressions 255′ in the track 251′ and can be slid laterally across the track 251′, thereby moving the tube holder assembly 313 horizontally. This allows a health worker to locate the tube 14 at either of the corners of the mouth, centrally in the mouth, or in some location therebetween.

Moreover, the rib 220 can be moved up and down on its two slides 240′ fit in the tracks 230′, so that a health worker can place the tube 14 more against the upper lip or more against the lower lip. For example, if a health worker notices that the right side of the mouth of the patient 11 is becoming irritated such that a sore may develop, the health worker can slide the upper portion 320 to the left so as to position the tube 14 at the - of the mouth (as in FIG. 11B). If the health worker then notices that the lower left side of the mouth is developing a sore, then the worker can move the slides 240′ up on the vertical guides 230′, such that the tube 14 moves up against the upper lip.

A preferred embodiment is fully and clearly described above so as to enable one having skill in the art to understand, make, and use the same. Those skilled in the art will recognize that modifications may be made to the description above without departing from the spirit of the invention, and that some embodiments include only those elements and features described, or a subset thereof. To the extent that modifications do not depart from the spirit of the invention, they are intended to be included within the scope thereof.

The invention claimed is:

1. An endotracheal tube mount comprising:
an armature including a central rib having opposed ends; and
a tube holder assembly mounted to the armature for horizontal movement along the rib between the opposed ends, the tube holder assembly comprising:
a shuttle which rides on the rib between the opposed ends; and
tube holder means coupled to the shuttle, for holding an endotracheal tube in a position below the rib;
wherein one of the armature and the tube holder assembly includes vertical adjustment means for adjusting the position of the endotracheal tube vertically with respect to the rib.

2. The endotracheal tube mount of claim 1, wherein the tube holder assembly includes the vertical adjustment means, which comprises:
a slide; and
a vertical guide mounted in the slide for vertical movement in the slide.

3. The endotracheal tube mount of claim 1, wherein the armature includes the vertical adjustment means comprising vertical guides, and the rib terminates at its opposed ends in slides which are mounted on the vertical guides for vertical movement of the rib with respect to the vertical guides.

4. The endotracheal tube mount of claim 1, wherein the tube holder assembly includes a cylindrical clamp for holding the endotracheal tube.

5. The endotracheal tube mount of claim 4, wherein the tube holder assembly further includes a strap and a crimp, the crimp comprising a socket and a hinged arm that moves between a locked position, in which the strap is retained in the crimp between the arm and the socket, and an unlocked position, in which the strap is loose from the crimp.

6. The endotracheal tube mount of claim 1, wherein the tube holder assembly includes a tie for holding the endotracheal tube.

7. The endotracheal tube mount of claim 1, wherein the armature further includes a pad carried at each of the opposed ends of the rib.

8. The endotracheal tube mount of claim 1, wherein:
the rib is formed with a track having spaced-apart depressions formed therein; and
the shuttle includes teeth which move into and out of the depressions to index the shuttle along the rib.

9. An endotracheal tube mount comprising:
an armature including a central rib having opposed ends; and
a tube holder assembly mounted to the armature for horizontal movement along the rib between the opposed ends, the tube holder assembly comprising:
a shuttle which rides on the rib between the opposed ends, the shuttle having a slide; and
tube holder means coupled to the shuttle, for holding an endotracheal tube in a position below the rib, the tube holder means having a vertical guide which is mounted in the slide for vertical movement therein to adjust the position of the endotracheal tube vertically with respect to the rib.

10. The endotracheal tube mount of claim 9, wherein the tube holder assembly includes a cylindrical clamp for holding the endotracheal tube.

11. The endotracheal tube mount of claim 10, wherein the tube holder assembly further includes a strap and a crimp, the crimp comprising a socket and a hinged arm that moves between a locked position, in which the strap is retained in the crimp between the socket and the arm, and an unlocked position, in which the strap is loose from the crimp.

12. The endotracheal tube mount of claim 9, wherein the tube holder assembly includes a tie for holding the endotracheal tube.

13. The endotracheal tube mount of claim 9, wherein the armature further includes a pad carried at each of the opposed ends of the rib.

14. The endotracheal tube mount of claim 9, wherein:
the rib is formed with a track having spaced-apart depressions formed therein; and
the shuttle includes teeth which move into and out of the depressions to index the shuttle along the rib.

15. An endotracheal tube mount comprising:
an armature including:
a central rib having opposed ends and a slide at each end; and
vertical guides, the slides mounted in the vertical guides for vertical movement of the rib with respect to the vertical guides;
a tube holder assembly mounted to the armature for horizontal movement along the rib between the opposed ends, the tube holder comprising:
a shuttle which rides on the rib between the opposed ends; and
tube holder means coupled to the shuttle, for holding an endotracheal tube in a position below the rib.

16. The endotracheal tube mount of claim 15, wherein the tube holder assembly includes a cylindrical clamp for holding the endotracheal tube.

17. The endotracheal tube mount of claim 16, wherein the tube holder assembly further includes a strap and a crimp, the crimp comprising a socket and a hinged arm that moves between a locked position, in which the strap is retained in the crimp between the socket and the arm, and an unlocked position, in which the strap is loose from the crimp.

18. The endotracheal tube mount of claim 15, wherein the tube holder assembly includes a tie for holding the endotracheal tube.

19. The endotracheal tube mount of claim 15, wherein the armature further includes a pad carried at each of the opposed ends of the rib.

20. The endotracheal tube mount of claim 15, wherein:
the rib is formed with a track having spaced-apart depressions formed therein; and
the shuttle includes teeth which move into and out of the depressions to index the shuttle along the rib.
